(19) United States
(12) Patent Application Publication
(10) Pub. No.: US 2025/0009968 A1
(43) Pub. Date: Jan. 9, 2025

(54) Vascular Access Device With Effective Needle Length Adjustment

(71) Applicant: Becton, Dickinson and Company, Franklin Lakes, NY (US)

(72) Inventors: Sajayesh Vijayachandran, Kerala (IN); Sudev GS, Kerala (IN)

(73) Assignee: Becton, Dickinson and Company, Franklin Lakes, NJ (US)

(21) Appl. No.: 18/708,737

(22) PCT Filed: Nov. 11, 2021
(86) PCT No.: PCT/US2021/058953

§ 371 Date: May 9, 2024

(51) Int. Cl. A61M 5/178; A61M 5/31; A61M 5/32; A61M 5/46

(52) U.S. Cl. CPC A61M 5/1782; A61M 5/3134; A61M 5/3202; A61M 5/3257; A61M 5/46

(57) ABSTRACT

Vascular access devices which have needle-length adjustment features for penetrating a vial of a stopper with the full needle length and for penetrating the skin of a patient with a desired needle length, which is less than the full needle length.

### TECHNICAL FIELD

The present disclosure relates to needle-length adjustable vascular access devices, and in particular the present disclosure relates to insulin syringes having needle length adjustment for penetrating both insulin vials and the skin of a patient.

### BACKGROUND

Syringes are commonly used in the art to withdraw medicament from a vial and injecting the medicament into the skin of a patient. Needle cannulas for syringes come in varying needle gauge and needle lengths depending on the intended use, the insertion depth into the skin of the patient and other factors. When withdrawing medicament from a vial, a thicker needle gauge can be used. Syringes can have an integrated, non-removable needle cannula affixed to the distal end of the barrel, or they can have a needleless connector which can attach to removable and interchangeable needle hubs.

Insulin syringes common in the art have a standard needle cannula length of 6 mm. The needle cannula of an insulin syringe can either be non-removably affixed to the syringe or can be attachable via a needle hub to a needleless connector of the insulin syringe. The 6 mm needle cannula length is used because a 6 mm needle cannula can penetrate a stopper of a standard insulin vial at any angle and reach the insulin in the vial for aspiration of insulin.

Insulin is commonly injected in the subcutaneous layer underneath the skin to ensure quick and uniform dispersion of insulin in the patient's body. Needle cannulas having a 6 mm length can pass through the subcutaneous layer into the muscle layer underneath it which is not desirable. To avoid this occurrence, a practitioner administering the insulin shot commonly has to pinch the site where the insulin shot/injection is to be taken to avoid the 6 mm needle cannula from penetrating into the muscle layer. This method can be a tedious process which can lead to patient discomfort, needlestick injuries and inconsistent results. In the alternative, interchangeable needle hubs can be used for first using a 6 mm needle cannula to withdraw medicament from a vial and subsequently using a smaller needle length to administer the medicament. This alternative results in further waste.

Thus, there is a need in the art to provide a single-use needle vascular access device which can adequately withdraw medicament from a vial and subsequently administer the medicament to the subcutaneous layer underneath the skin.

### SUMMARY

A first aspect of the present disclosure relates to a vascular access device having a cylindrical base, a tab, and a moveable collar. The cylindrical base has a proximal end and a distal end. The proximal end of the cylindrical base has a proximal wall and an elongate tip extending from the proximal wall, the elongate tip having one or more longitudinal slots. The cylindrical base further has an outer cylindrical wall and an outer cavity between the outer cylindrical wall and the elongate tip, the outer cylindrical wall has a cutout extending from the distal end to the proximal wall. The tab is connected to the distal end of the outer cylindrical wall having a living hinge connecting the tab to the outer cylindrical wall. The tab is configured to pivot about the living hinge. The tab covers the cutout of the cylindrical base in a closed position. The tab further includes a crescent-shaped cam having a stem and a distal peak, the distal peak extending in a distal direction in the closed position. The moveable collar disposed within the outer cavity, the moveable collar having a proximal end and a distal end, the distal end comprising one or more snap locks configure to travel within the one or more longitudinal slots, the moveable collar further comprising a clearance cutout extending from the distal end to a distance along the moveable collar, the clearance cutout having a cam-contact surface.

In some embodiments, the elongate tip includes a proximal portion and a distal portion, the distal portion has an outer surface. The elongate tip further includes an aperture extending therethrough, the one or more longitudinal slots extending on the proximal portion.

In some embodiments, a contact surface is formed between the proximal portion and distal portion of the elongate tip. In some embodiments, a sterile cap is inserted over the elongate tip and is in contact with the contact surface.

In some embodiments, the living hinge includes one or more longitudinal protrusions configured to create a snap fit with corresponding slots disposed on the proximal wall.

In some embodiments, the proximal wall has a cavity for receiving a distal end of a vascular access device.

In some embodiments, the moveable collar includes a distal flange, the distal flange configured to abut the distal end of the outer cylindrical wall upon full advancement of the moveable collar within the cylindrical base. In some embodiments, the moveable collar has an initial position, an aspiration position and an injection position.

In some embodiments, a needle cannula of the vascular access device is fully covered by the moveable collar in the initial position. In some embodiments, a needle cannula of the vascular access device is fully exposed by a distance Df in the aspiration position. In some embodiments, the distance Df is 6 mm.

In some embodiments, a needle cannula of the vascular access device is partially exposed by a distance Dn in the injection position. In some embodiments, the distance Dn is 4 mm.

In some embodiments, advancement of the moveable collar from the initial position causes the cam-contact surface of the moveable collar to abut against the crescent-shaped cam of the tab, causing the tab to pivot outwardly about the living hinge.

In some embodiments, depressing of the tab causes the moveable collar to advance to the injection position.

A second aspect of the disclosure relates to a vascular access device having a cylindrical base, a cammed slide disposed over the cylindrical base, and a biasing element disposed between the distal wall of the cammed slide and the proximal wall of the cylindrical base.

In some embodiments, the cylindrical base has a proximal end, a distal end, a proximal wall and an elongate tip extending from the proximal wall. In some embodiments, the elongate tip further includes an aperture extending therethrough. In some embodiments, the cylindrical base has an outer cylindrical wall and a cavity defined by the elongate tip, outer cylindrical wall and the proximal wall. In some embodiments, the cylindrical base also has a cam path disposed on an outer surface of the outer cylindrical wall, the distal end having a snap-element.

In some embodiments, the cammed slide is disposed over the cylindrical base having a proximal end, a distal end and an inner surface. The distal end has a distal wall. The inner surface has a peg extending inwardly and a slot, the peg is configured to travel within the cam path.

The biasing element is configured to apply a biasing force.

In some embodiments, the peg advances along the cam path between at least three points, a first point defining an initial position, a second point defining an aspiration position and a third point defining an injection position, wherein a needle cannula is fully covered in the initial position, the needle cannula is fully exposed by a distance D1 in the aspiration position and the needle cannula is partially exposed by a distance D2 in the injection position. In some embodiments, the distance D1 is 6 mm and the distance D2 is 4 mm.

In some embodiments, the proximal wall includes a cavity for receiving a distal end of a vascular access device.

In some embodiments, the distal wall of the cammed slide includes an inner collar extending in a proximal direction.

In some embodiments, the peg is positioned at a right angle plane relative to the slot and the peg is located proximally to the slot.

In some embodiments, the peg can advance in a clockwise or counterclockwise direction.

In some embodiments, the snap-element includes a flat surface and a sloped surface opposite the sloped surface.

In some embodiments, the biasing element is configured to continuously hold the cammed slide in the initial position such that the needle cannula is covered.

In some embodiments, depression of the cammed slide in a proximal direction relative to the cylindrical base with a depression force greater than the biasing force causes the cammed slide to reversibly advance to the aspiration position. In some embodiments, depression of the cammed slide in a proximal direction relative to the cylindrical base with a depression force greater than the biasing force causes the cammed slide to non-reversibly advance to the aspiration position.

In some embodiments, withdrawing the depression force causes the cammed slide to enter the injection position.

In some embodiments, the cammed slide is held in the injection position by the snap-element of the cylindrical base interdigitating with the slot of the cammed slide. In some embodiments, the cammed slide returns to the initial position upon application of a rotational release force.

### BRIEF DESCRIPTION OF THE DRAWINGS

FIG. 1 illustrates a side view of a vascular access device attached to a syringe in accordance with one or more embodiments of the present disclosure;

FIG. 2 illustrates an exploded side view of a vascular access device in accordance with one or more embodiments of the present disclosure;

FIG. 3 illustrates a detailed cross-sectional view a vascular access device in accordance with one or more embodiments of the present disclosure;

FIGS. 4 and 5 illustrate a perspective view of a base of a vascular access device in accordance with one or more embodiments of the present disclosure;

FIGS. 6 and 7 illustrate a perspective view of a moveable insert of a vascular access device in accordance with one or more embodiments of the present disclosure;

FIG. 8 illustrates an initial position of a vascular access device in accordance with one or more embodiments of the present disclosure;

FIG. 9 illustrates an aspiration position of a vascular access device in accordance with one or more embodiments of the present disclosure;

FIG. 10 illustrates an injection position of a vascular access device in accordance with one or more embodiments of the present disclosure;

FIG. 11 illustrates a vascular access device in the initial position in accordance with one or more embodiments of the present disclosure;

FIGS. 12 and 13 illustrate a vascular access device in the aspiration position in accordance with one or more embodiments of the present disclosure;

FIG. 14 illustrates the vascular access device in the injection position in accordance with one or more embodiments of the present disclosure;

FIG. 15 illustrates a side view of a vascular access device attached to a syringe in accordance with one or more embodiments of the present disclosure;

FIG. 16 illustrates an exploded side view of a vascular access device in accordance with one or more embodiments of the present disclosure;

FIG. 17 illustrates a detailed cross-sectional view a vascular access device in accordance with one or more embodiments of the present disclosure;

FIG. 18 illustrates a perspective view of a base of a vascular access device in accordance with one or more embodiments of the present disclosure;

FIG. 19 illustrates a side view of a base of a vascular access device in accordance with one or more embodiments of the present disclosure;

FIG. 20 illustrates a detailed view a base of a vascular access device in accordance with one or more embodiments of the present disclosure;

FIGS. 21 and 22 illustrate perspective views of a slide a vascular access device in accordance with one or more embodiments of the present disclosure;

FIG. 23 illustrates an initial position of a vascular access device in accordance with one or more embodiments of the present disclosure;

FIG. 24 illustrates an aspiration position of a vascular access device in accordance with one or more embodiments of the present disclosure;

FIG. 25 illustrates an injection position of a vascular access device in accordance with one or more embodiments of the present disclosure; and,

FIGS. 26A through 26G illustrate a method of use of a vascular access device in accordance with one or more embodiments of the present disclosure.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

For purposes of the description hereinafter, the terms “proximal”, “distal”, “longitudinal”, and derivatives thereof shall relate to the disclosure as it is oriented in the drawing figures. However, it is to be understood that the disclosure may assume alternative variations and step sequences, except where expressly specified to the contrary. It is also to be understood that the specific devices and processes illustrated in the attached drawings, and described in the following specification, are simply exemplary embodiments of the disclosure. Hence, specific dimensions and other physical characteristics related to the embodiments disclosed herein are not to be considered as limiting.

As used herein, the use of “a,” “an,” and “the” includes the singular and plural.

As used herein, the term “Luer connector” refers to a connection collar that is the standard way of attaching syringes, catheters, hubbed needles, IV tubes, etc. to each other. The Luer connector consists of one or more interlocking tubes, slightly tapered to hold together with just a simple pressure/twist fit/friction fit. Luer connectors can optionally include an additional outer rim of threading, allowing them to be more secure. The Luer connector can interlock and connect to the end located on the vascular access device (VAD). A Luer connector comprises a distal end, a proximal end, an irregularly shaped outer wall, a profiled center passageway for fluid communication from the chamber of the barrel of a syringe to the hub of a VAD. A Luer connector also has a distal end channel that releasably attaches the Luer connector to the hub of a VAD, and a proximal end channel that releasably attaches the Luer connector to the barrel of a syringe. As used herein, the term “Luer connector” refers to a male luer connector or a female luer connector.

As used herein, the term “medical device” refers to common medical devices having threaded or interlocking connections, the connections having corresponding mating elements. By way of example but not limitation, a syringe may have a threaded connection which releasably interlocks with a secondary medical device such as a needless connector of a catheter, an IV line and the like. The threaded connection may include a lumen defining a fluid path surrounded by a protruding wall having the threaded means for attaching to the secondary medical device.

As would be readily appreciated by skilled artisans in the relevant art, while descriptive terms such as “thread”, “taper”, “tab”, “wall”, “proximal”, “side”, “distal” and others are used throughout this specification to facilitate understanding, it is not intended to limit any components that can be used in combinations or individually to implement various aspects of the embodiments of the present disclosure.

Embodiments of the present disclosure are directed to vascular access devices which have needle-length adjustment features for penetrating a vial of a stopper with the full needle length and for penetrating the skin of a patient with a desired needle length, which is less than the full needle length. The vascular access devices described herein can be integral to a syringe barrel or can be attachable to a needless connector of a syringe barrel. The vascular access devices described herein can be integral to any medical device for withdrawing or injecting medicament from a vial and injecting into the skin of a patient. In some embodiments, the vascular access devices are either integral or removably connectable to insulin syringes.

In insulin administration, needle cannulas commonly have a length of 6 mm because such a length is capable of penetrating a stopper of a standard insulin vial at any angle and reach the insulin in the vial for aspiration of insulin. The same needle cannula used to withdraw medicament from a vial is subsequently used to inject the medicament into the skin of a patient. For insulin administration, the insulin medicament is administered in the subcutaneous layer underneath the skin to ensure quick and uniform dispersion of insulin in the patient's body. However, a cannula length of 6 mm will result in passing through the subcutaneous layer into the muscle layer underneath it which is not desirable. Embodiments of the present disclosure have passive features which enable a needle cannula of (by way of example but not limitation, a 6 mm needle cannula) to be fully inserted into a vial at any angle but only partially inserted into the skin of a patient at a desired skin depth (by way of example but not limitation, into the subcutaneous layer underneath the skin).

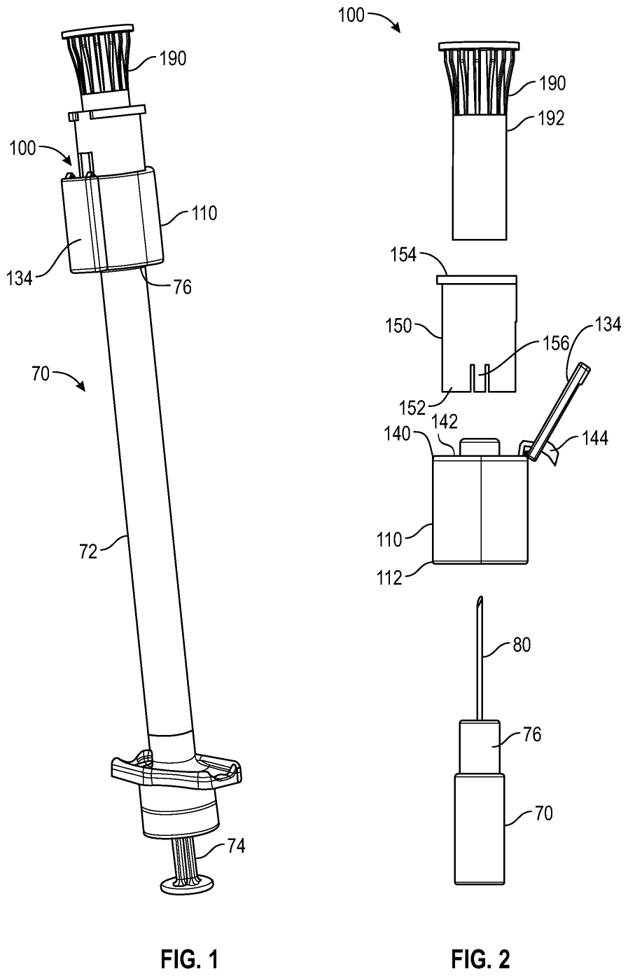

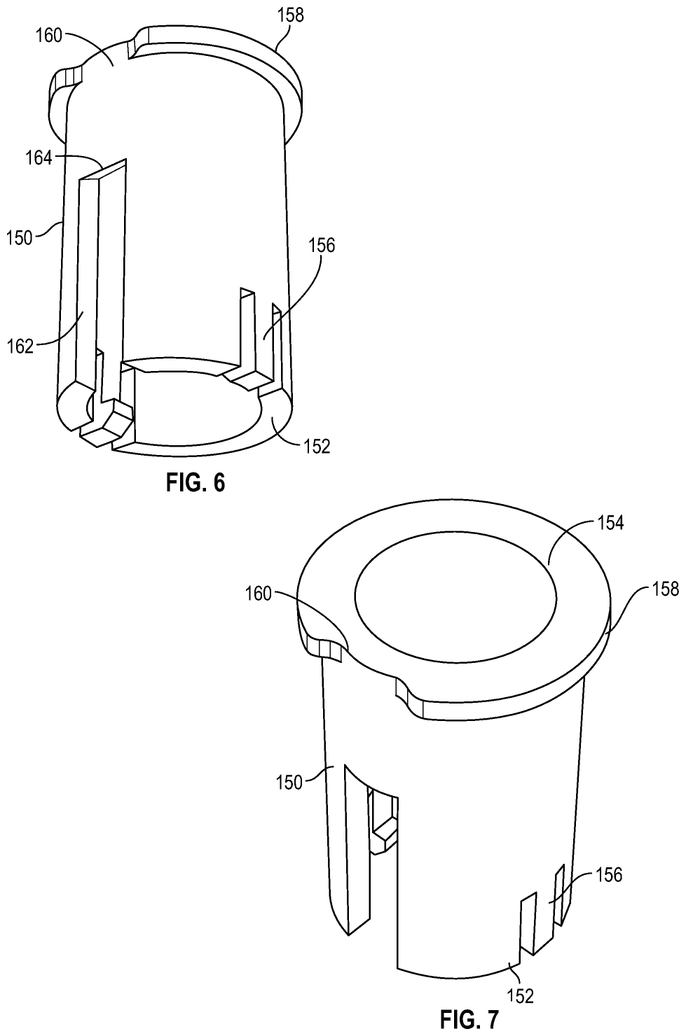

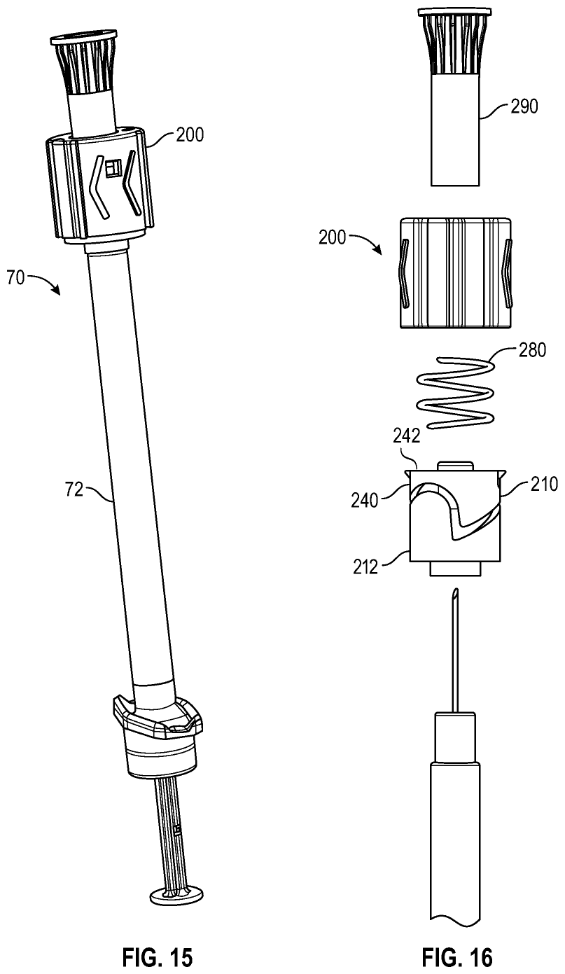

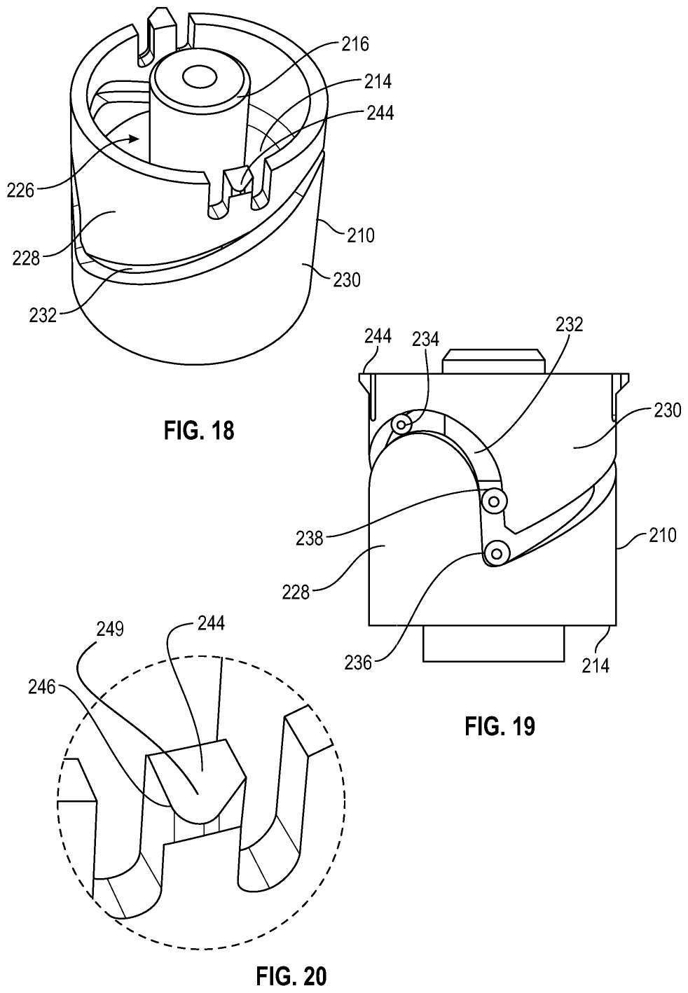

FIGS. 1 through 14 illustrate an embodiment of a vascular access device 100 having a sliding mechanism for partially inserting a fixed-length needle cannula into the skin of a patient at a desired skin depth. FIGS. 15 through 26 illustrate an embodiment of a vascular access device 200 having a cammed slide for partially inserting a fixed-length needle cannula into the skin of a patient at a desired skin depth.

Throughout the figures (FIGS. 1-26), a conventional vial 40 is illustrated. The vial 40 has a cylindrical body 42 having a cavity for storing medicament. The cylindrical body 42 has a closed base and a neck 44 opposite the closed base. The neck 44 has an opening 46 into which a rubber stopper 50 is inserted, creating a seal. In some embodiments, the neck 44 further comprises a flange 48. In some embodiments, the rubber stopper 50 has a trapezoidal shape. In some embodiments, the rubber stopper 50 has a width substantially equal to a width of the flange 48. As shown in the figures, a needle cannula penetrates a top surface 52 of the rubber stopper 50 to withdraw medicament from the vial 40.

Throughout the figures (FIGS. 1-26), a conventional syringe 70 is illustrated. The syringe 70 includes a barrel 72 having a distal end 76. The syringe further includes a plunger rod 74 which can be partially withdrawn from the barrel 72, causing an internal negative pressure within the barrel 72 such that fluid or medicament can be drawn into the barrel 72. Likewise, the plunger rod 74 can be depressed into the barrel 72, causing an internal positive pressure buildup such that fluid or medicament can be dispelled out of the barrel 72. In some embodiments, the distal end 76 is integral to one or more of the vascular access devices (100, 200) as explained in further detail below. In some embodiments, the distal end 76 is removably attachable to one or more of the vascular access devices (100, 200) as explained in further detail below. In some embodiments, the distal end 76 is removably attachable to one or more of the vascular access devices (100, 200) by a needleless connection.

As shown in FIGS. 1 through 14, the vascular access device 100 in accordance with one or more embodiments comprises a cylindrical base 110 and a moveable collar 150. FIG. 1 illustrates an assembled view of the vascular access device 100 onto the syringe 70 and FIG. 2 illustrates an exploded view of the vascular access device. As shown in FIGS. 1 and 2, in some embodiments the vascular access device further includes a sterile cap 190. As explained in further detail below, the moveable collar 150 is translatable proximally and distally within the cylindrical base to either reduce or increase the effective length of the needle cannula 80.

As shown in FIGS. 2 through 4, the base 110 has a cylindrical shape comprising a proximal end 112 and a distal end 140 having a distal surface 142. The proximal end 112 comprises a proximal wall 114, the proximal wall 114 having an elongate tip 116 extending in a distal direction. In some embodiments, the elongate tip 116 extends distally past the distal end 140 and distal surface 142. In some embodiments, the elongate tip 116 extends to the distal end 140 and shares a common distal surface 142 with the distal end 140. In some embodiments, a cavity extends from the proximal wall 114 in a distal direction, the cavity 120 extending a distance into the elongate tip. The cavity 120 is configured to receive the distal end 76 of the syringe 70. The elongate tip 116 includes aperture 122 extending through the elongate tip 116 and in fluid communication with the cavity 120.

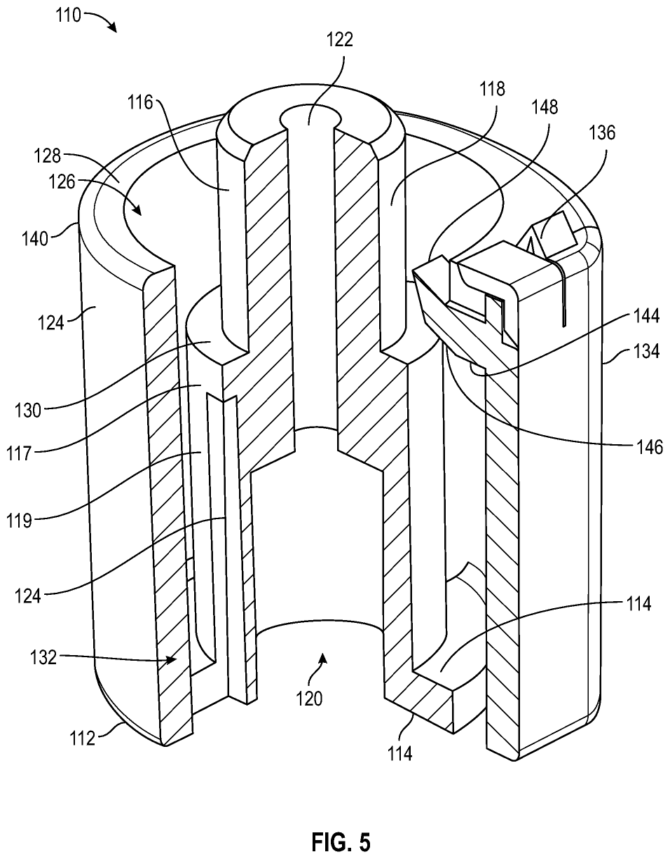

As best shown in FIG. 5, the elongate tip 116 has a proximal portion 117 and a distal portion 118, the proximal portion 117 adjacent to the proximal wall 114 and having a larger diameter than the distal portion 118. As shown in FIG. 5, the cavity 120 extends within the proximal portion 117. The proximal portion 117 has an outer surface 119 having one or more longitudinal slots 124. The one or more longitudinal slots 124 extend from the proximal wall 114 to a distance distally along the proximal portion 117 of the elongate tip 116. The one or more longitudinal slots 124 are configured to interdigitate with snap locks 156 of the moveable collar 150 as explained in further detail below. The distance of the one or more longitudinal slots 124 provide a longitudinal travel distance limit for the moveable collar 150 as the snap locks 156 can travel from the proximal wall 114 to the distance of the one or more longitudinal slots 124.

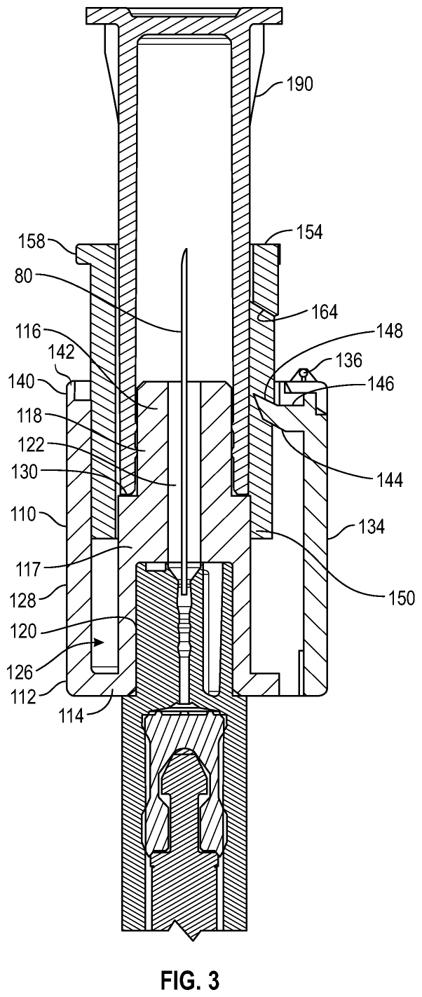

As best shown in FIG. 5, an outer cavity 126 of the base 110 separates the elongate tip 116 from an outer cylindrical wall 128 such that the elongate tip 116 is surrounded by the outer cylindrical wall 128. The outer cylindrical wall 128 extends from the proximal wall 114 to the distal end 140. Stated differently, the outer cylindrical wall 128 surrounds the outer cavity 126 and the elongate tip 116. Due to the elongate tip 116 having a proximal portion 117 with a larger diameter than the diameter of the distal portion 118, a contact surface 130 is formed between the proximal portion 117 and distal portion 118 of the elongate tip. As shown in FIG. 3, the sterile cap 190, when placed over the elongate tip 116, is seated against the contact surface 130. The sterile cap 190 has a hollow cylindrical body which is configured to create an interference fit with the distal portion 118 of the elongate tip 116. Furthermore, the sterile cap 190 has an outer diameter which is equal to or less than the diameter of the proximal portion 117 of the elongate tip 116 such that an outer surface 192 of the sterile cap 190 does not protrude or project laterally beyond the proximal portion 117. As explained in further detail below, the moveable collar 150 can travel longitudinally in a proximal and distal direction, and the moveable collar 150 can travels between the outer cylindrical wall 128 and the combination outer surface 192 of the sterile cap 190 and proximal portion 117 of the elongate tip 116.

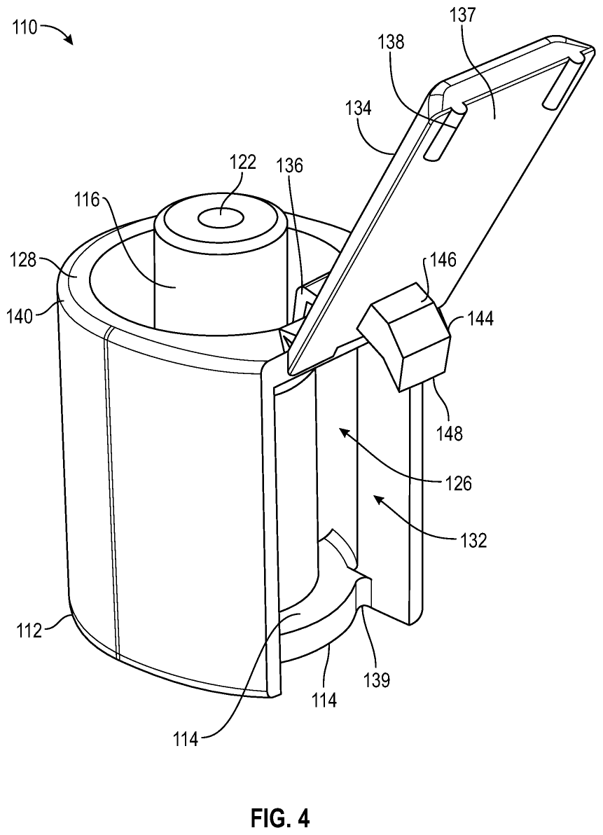

As best shown in FIG. 4, the outer cylindrical wall 128 has a cutout 132 extending a distance proximally from the distal surface 142 to at least partially the length of the outer cylindrical wall 128. In some embodiments, the cutout 132 extends to the proximal wall 114. The cutout 132 is at least partially covered by a tab 134 connected to the distal surface 142 of the distal end 140 and outer cylindrical wall 128 by a living hinge 136 providing a pivot for the tab 134 to rotate relative to the distal surface 142.

FIG. 4 illustrates the tab 134 in an open position and FIG. 5 illustrates the tab 134 in a closed position. As shown, the tab 134 substantially covers the cutout 132. In some embodiments, an inner surface 137 of the tab 134 has longitudinal protrusions 138 configured to create an interference or snap fit with corresponding slots 139 in the proximal wall 114. As best shown in FIGS. 4 and 5, the inner surface 137 of the tab 134 further comprises a distally positioned crescent-shaped cam 144. A stem 146 of the crescent-shaped cam 144 extends medially inward and a distal peak 148 of the crescent-shaped cam 144, when in the closed position as shown in FIG. 3, is directed distally. As explained in further detail below, advancement of the moveable collar 150 in the proximal direction causes a contact surface of the moveable collar 150 to push the distal peak 148 causing the tab 134 to rotate about the living hinge 136 and enter the open position as shown in FIG. 4. As explained in further detail below, the tab 134 provides a visual indication to a practitioner of the relative position of the moveable collar 150.

As shown in FIGS. 3, 6 and 7, the moveable collar 150 comprises a hollow cylindrical body having a proximal end 152 and a distal end 154. From the distal end 154 extends a flange 158 configured to seat against the outer cylindrical wall 128. In some embodiments, the flange 158 further comprises a relief cutout 160 such that when the flange 158 is seated against the outer cylindrical wall 128, the flange 158 does not interfere with the tab 134 and living hinge 136. The proximal end 152 comprises one or more snap locks 156 which create a snap-fit with the one or more longitudinal slots 124 of the elongate tip 116. The one or more snap locks 156 allow for the moveable collar 150 to travel in the proximal and distal direction, but prevent the moveable collar 150 to be fully removed from the cylindrical base 110. In some embodiments, a clearance cutout 162 extends from the proximal end 152 to a distance along the moveable collar 150. The clearance cutout 162 is configured to not interfere with the crescent-shaped cam 144.

As shown in FIG. 3, the clearance cutout 162 comprises a cam-contact surface 164. As the moveable collar 150 is advanced proximally within the outer cavity 126, the cam-contact surface 164 comes into contact with the distal peak 148 of the crescent-shaped cam 144, causing the tab 134 to pivot about the living hinge 136.

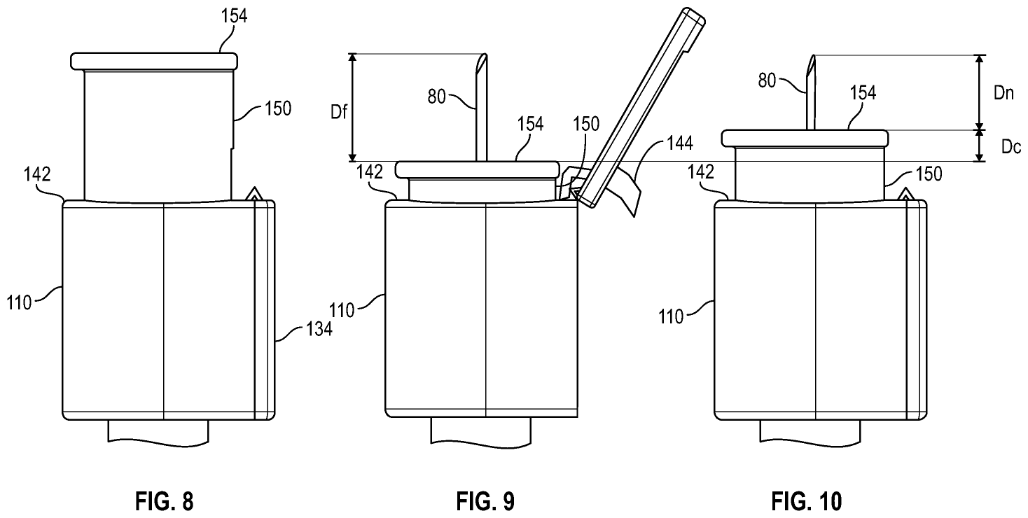

FIGS. 8 through 10 illustrate an initial position, an aspiration position and an injection position of the vascular access device. As shown in FIG. 8, in the initial position the tab 134 is closed and the moveable insert 150 fully convers the needle cannula (not shown). As shown in FIG. 9, advancement of the moveable insert 150 in the proximal direction causes the cam-contact surface 164 comes into contact with the distal peak 148 of the crescent-shaped cam 144, causing the tab 134 to pivot about the living hinge 136. This is the aspiration position, in which the needle cannula 80 is fully exposed a distance Df. In some embodiments, the distance Df is 6 mm. The distance Df of 6 mm is preferable for aspiration of an insulin vial. By the tab 134 pivoting outward, the user has a visual indication that the vascular access device 100 is in the aspiration position. To bring the vascular access device into the injection position, the user closes the tab 134 to bring the tab to the closed position. The crescent-shaped cam 144 pushes the moveable collar 150 in a distal direction by a distance Dc, thereby reducing the distance Df of the needle cannula 80 to an effective distance Dn. In some embodiments, where the distance Df is 6 mm, the distance Dc is 2 mm and the effective needle cannula 80 distance Dn is 4 mm. A 4 mm will penetrate the subcutaneous layer of the skin 20 of a patient, but not penetrate into the muscle layer.

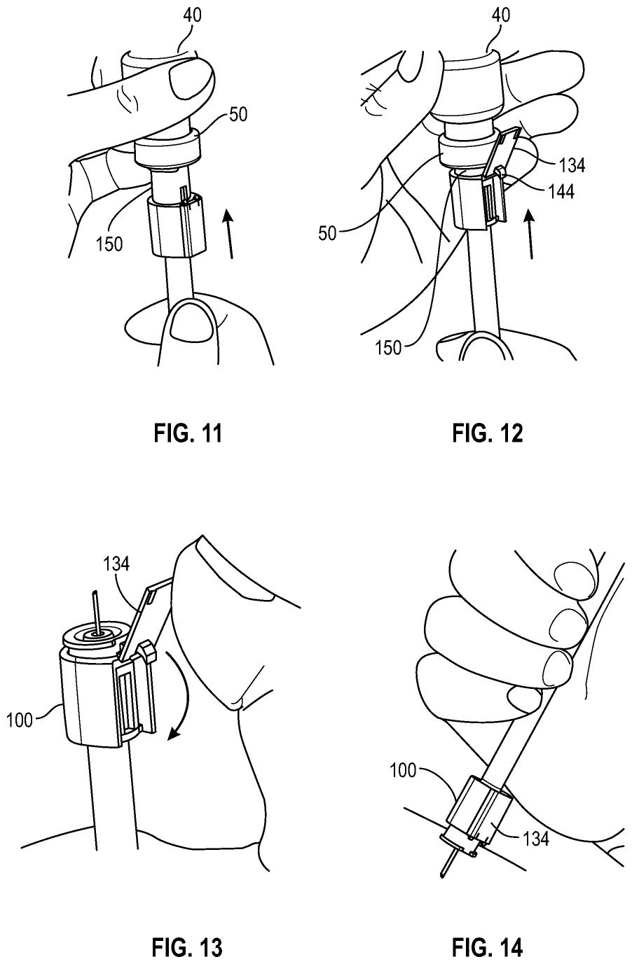

FIG. 11 illustrates the vascular access device 100 in the initial position. FIGS. 12 and 13 illustrate the vascular access device 100 in the aspiration position, and FIG. 14 illustrates the vascular access device 100 in the injection position.

As shown in FIGS. 11 and 12, a method of use of the vascular access device 100 comprises the steps of: removing the sterile cap 190, pushing the moveable insert 150 against the rubber stopper 50 of the vial 40 until the moveable insert 150 biases against the crescent-shaped cam 144, thus causing the tab 134 to pivot outward. As shown in FIGS. 13 and 14, the method further comprises the steps of removing the vascular access device 100 from the vial 40, and pushing the tab 134 inward, bringing the vascular access device into the injection position. The vascular access device 100 can then be inserted into the skin of a patient in the injection position.

As shown in FIGS. 15 through 25, the vascular access device 200 in accordance with one or more embodiments comprises a cylindrical base 210, a cammed slide 250 and a biasing element 280. FIG. 15 illustrates an assembled view of the vascular access device 200 onto the syringe 70 and FIG. 16 illustrates an exploded view of the vascular access device 200. As shown in FIGS. 15 and 16, in some embodiments the vascular access device further includes a sterile cap 290. As explained in further detail below, the cammed slide 250 is translatable proximally and distally over the cylindrical base to either reduce or increase the effective length of the needle cannula 80.

As shown in FIGS. 16 through 20, the base 210 has a cylindrical shape comprising a proximal end 212 and a distal end 240 having a distal surface 242. In some embodiments, a proximal wall 214 is located at the proximal end 212. In some embodiments, the proximal wall 214 is located a distance from the proximal end 212. The proximal wall 214 has an elongate tip 216 extending in a distal direction. In some embodiments, the elongate tip 216 extends distally past the distal end 240 and distal surface 242. In some embodiments, the elongate tip 216 extends to the distal end 240 and shares a common distal surface 242 with the distal end 240. In some embodiments, a cavity extends from the proximal wall 214 in a distal direction, the cavity 220 extending a distance into the elongate tip. The cavity 220 is configured to receive the distal end 76 of the syringe 70. The elongate tip 216 includes aperture 222 extending through the elongate tip 216 and in fluid communication with the cavity 220.

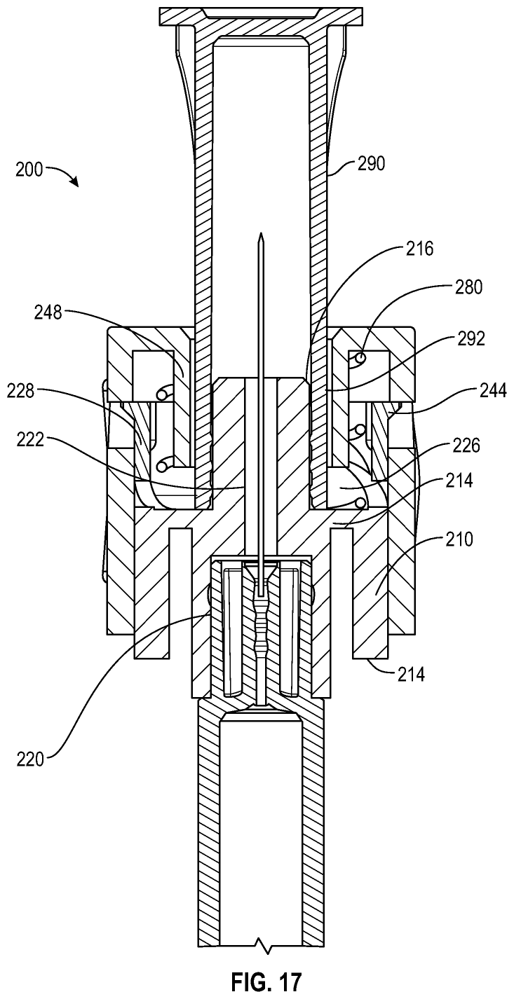

As best shown in FIG. 17, an outer cavity 226 of the base 210 separates the elongate tip 216 from an outer cylindrical wall 228 such that the elongate tip 216 is surrounded by the outer cylindrical wall 228. The outer cylindrical wall 228 extends from the proximal wall 214 to the distal end 240. Stated differently, the outer cylindrical wall 228 surrounds the outer cavity 226 and the elongate tip 216. As shown in FIG. 17, the sterile cap 290, when placed over the elongate tip 216, is seated against the proximal wall 214. The sterile cap 290 has a hollow cylindrical body which is configured to create an interference fit with the elongate tip 216.

As shown in FIGS. 17-20, the outer cylindrical wall 228 comprises a cam path 232 disposed an outer surface 230 of the outer cylindrical wall 228. The cam path 232 in some embodiments is a S-shaped cutout in the thickness of the outer cylindrical wall 228 configured to receive a peg 252 of the cammed slide 250 as explained in further detail below. The cam path 232 extends around the outer cylindrical wall 228 and (as best shown in FIG. 19) comprises at least three points (234, 236, 238) in which the peg 252 of the cammed slide 250 at least partially stops within. The first point 234 defining an initial position, the second point 236 defining an aspiration position, the third point 238 defining an injection position. As explained in further detail below, the peg 252 of the cammed slide 250 can travel between the at least three points (234, 236, 238) in a clockwise or counterclockwise direction. Stated differently, the positions are reversable and thus the peg 252 of the cammed slide 250 can travel from the first point 234 (initial position) to the third point 238 (injection position) through the second point 236 (injection position), and can also reversibly travel from the third point 238 (injection position) to the first point 234 (initial position) through the second point 236 (injection position).

The outer cylindrical wall 228 further comprises a snap-element 244 extending outward or laterally from the outer cylindrical wall 228 at the distal end 240. In some embodiments, the snap-element 244 includes a flat surface 246 opposite a sloped surface 249. In embodiments where the cammed slide 250 is traveling between the at least three points (234, 236, 238) in a counterclockwise direction, the flat surface 246 is ahead of the sloped surface 249 (as shown in FIG. 20). In embodiments where the cammed slide 250 is traveling between the at least three points (234, 236, 238) in a clockwise direction, the sloped surface 249 is ahead of the flat surface 246. As explained in further detail below, the snap-element is configured to interdigitate with a slot of the cammed slide. The snap-element 244 and slot of the cammed slide 250 is configured to create a soft stop where the user has to apply additional force for the cammed slide to 250 to advance to an adjacent position of the least three points (234, 236, 238).

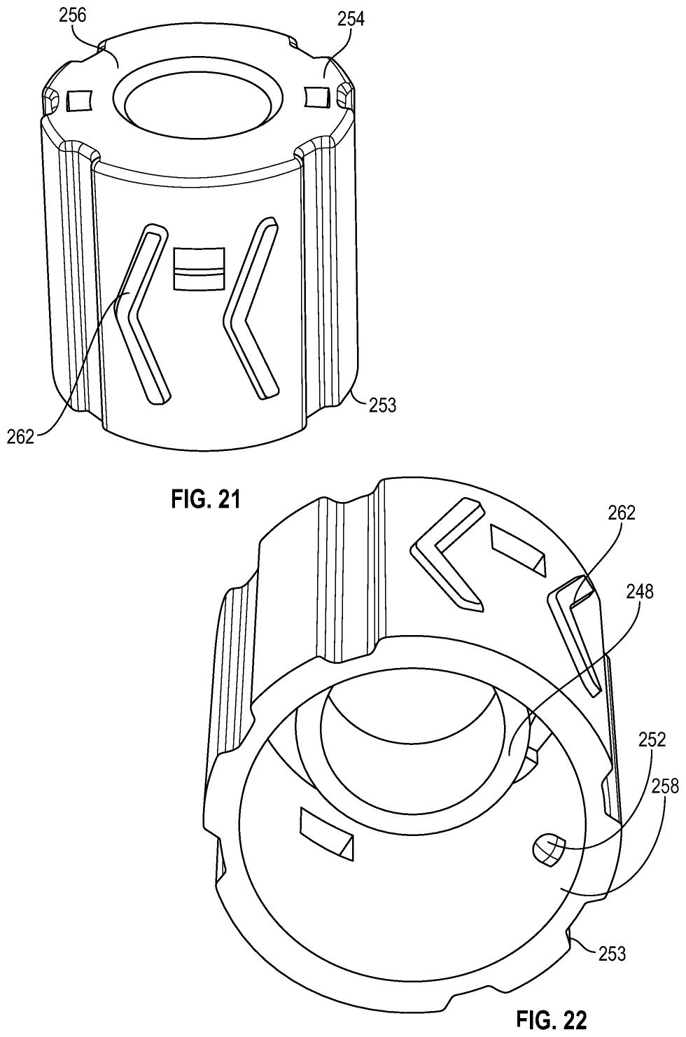

As shown in FIGS. 21 and 22, the cammed slide 250 comprises a hollow cylindrical body having a proximal end 253 and a distal end 254. The distal end 254 has a distal wall 256 comprising an inner collar 248 extending in a proximal direction relative to the distal end 254 and distal wall 256. The inner collar 248 is has an inner diameter to receive the sterile cap 290. An inner surface 258 of the hollow cylindrical body of the cammed slide 250 comprises the peg 252 extending inwardly and a slot 260. As shown in FIG. 22, the peg 252 is positioned at a right angle plane relative to the slot 260, and the peg 252 is located proximally to the slot 260.

The cammed slide 250 is positioned over the cylindrical base 210 such that the peg 252 is within the cam path 232. As the peg 252 rotates within the cam path 232, the entire cammed slide 250 translates both rotationally around the cylindrical base 210 and between the at least three points (234, 236, 238) in a clockwise or counterclockwise direction. Stated differently, the positions are reversable and thus the peg 252 of the cammed slide 250 can travel from the first point 234 to the third point 238 through the second point 236, and can also reversibly travel from the third point 238 to the first point 234 through the second point 236.

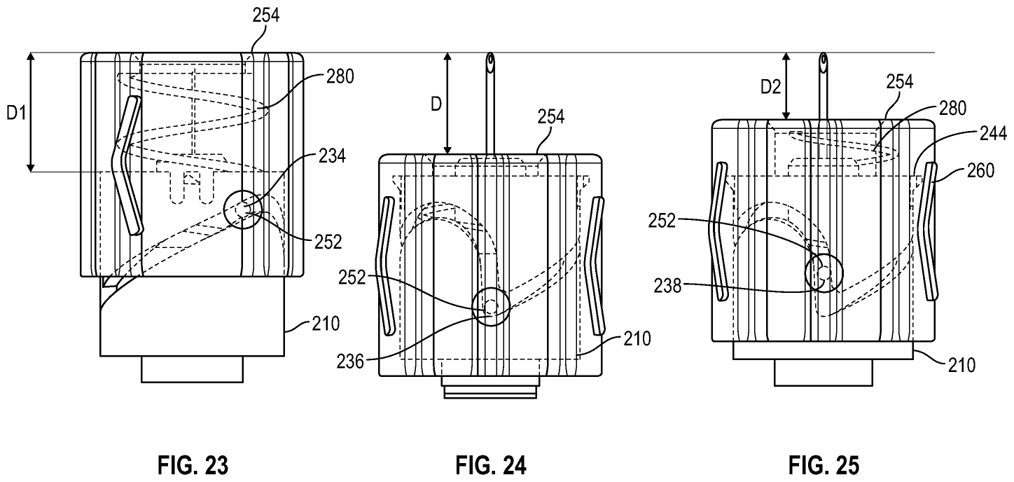

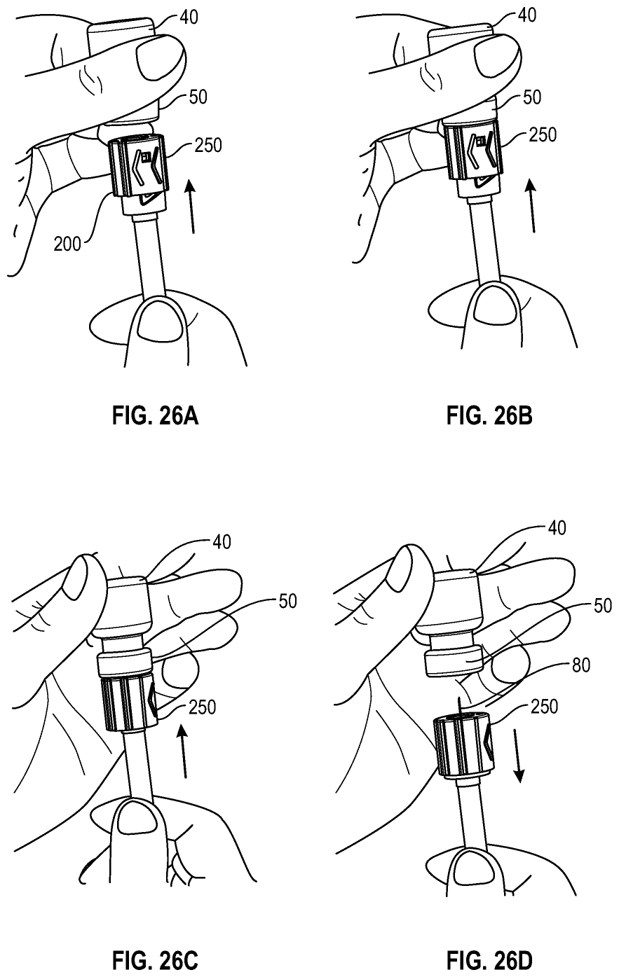

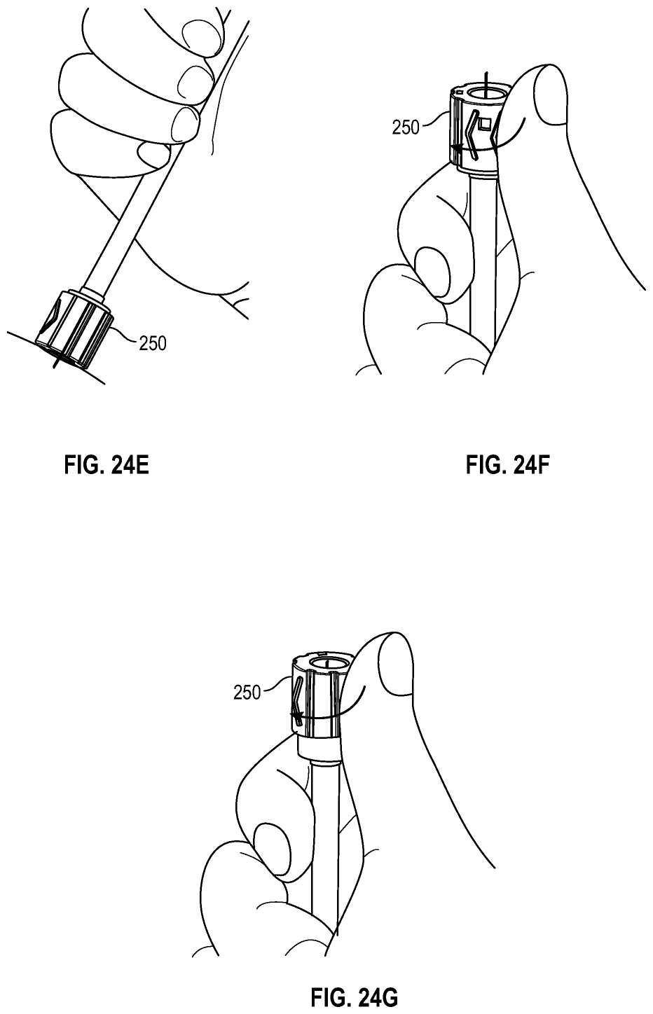

FIGS. 23 through 25 illustrate the cammed slide 250 at least partially disposed over the cylindrical base 210, and the peg 252 of the cammed slide 250 disposed and rotationally advanceable within the cam path 232 of the cylindrical base 210. FIG. 23 illustrates the initial position, FIG. 24 illustrates the aspiration position and FIG. 25 illustrates the injection position.

As shown in FIG. 23, the first point 234 is located distally from both the second point 236 and third point 238. The location of the first point 234 causes the cammed slide 250 to be in a fully extended position where the cammed slide 250 fully covers the needle cannula 80. The distal end 254 of the cammed slide 250 is at a distance D1 from the distal end 240 of the cylindrical base 210. The distance D1 (essentially the projection distance of the distal end 254 of the cammed slide 250 past the distal end 240 of the cylindrical base 210) fully covers the needle cannula 80.

As shown in FIG. 24, the second point 236 located proximally from both the first point 234 and the third point 238. The location of the second point 236 causes the cammed slide 250 to be in a fully retracted position where the needle cannula 80 is fully exposed by a distance D. In some embodiments, in the aspiration position the distal wall 256 abuts the distal end 240 of the cylindrical base 210. In some embodiments, in the aspiration position, the distance D is 6 mm, which is the standard needle size to aspirate medicament from an insulin vial.

As shown in FIG. 25, the third point 238 is located proximal to the first point 234 and distal to the second point 236. The location of the third point 238 causes the cammed slide 250 to be in a partially retracted position where the needle cannula 80 is partially exposed by a distance D2. In some embodiments, the distance D2 is configured to penetrate the subcutaneous layer of the skin of a patient, but not penetrate into the muscle layer. In some embodiments, where the distance D is 6 mm, the distance D2 is 4 mm. Furthermore, at the injection position, the slot 260 of the cammed slide 250 interdigitates with the snap-element 244 such that the cammed slide 250 cannot further rotate or translate proximally or distally without a force necessary to overcome the snap-element 244 as explained in further detail below.

As shown in FIGS. 16, 17, 23 and 25, the biasing element 280 is biased against the proximal wall 214 of the cylindrical base 210 and the distal wall 256 of the cammed slide 250. The biasing element is disposed within the outer cavity 226 and more specifically between the inner collar 248 of the cammed slide 250 and the outer cylindrical wall 228 of the cylindrical base 210. The biasing element 280 applies a biasing force against the cammed slide 250 in the distal direction relative to the cylindrical base 210. As previously stated, the positions are reversable and thus the peg 252 of the cammed slide 250 can travel from the first point 234 to the third point 238 through the second point 236, and can also reversibly travel from the third point 238 to the first point 236 through the second point 236. In some embodiments, the biasing element 280 is configured to continuously hold the cammed slide 250 in the initial position such that the needle cannula 80 is always covered. Depression of the cammed slide 250 in a proximal direction relative to the cylindrical base 210 with a depression force greater than the biasing force will cause the cammed slide 250 to reversibly advance to the aspiration position.

In some embodiments, the cammed slide 250 and vascular access device 200 will remain in the aspiration position until a practitioner or user advances the cammed slide 250 in a distal direction against a surface such as rubber stopper of a vial with a depression force. The vascular access device 200 remains in the aspiration position so long as the cammed slide 250 is depressed against the surface with the depression force, fully exposing the needle cannula 80 only while the needle cannula 80 is within the rubber stopper. Withdrawal of the cammed slide 250 from the surface causes the cammed slide 250 and vascular access device 200 to enter the injection position. As previously stated, the cammed slide 250 is held in the injection position by the snap-element 244 of the cylindrical base 210 interdigitating with the slot 260 of the cammed slide 250. Due to the cammed slide 250 being held such that the needle cannula is exposed by a distance D2 (as shown in FIG. 25), the user or practitioner can insert the needle cannula 80 into the skin of a patient without further movement of the cammed slide 250. Stated differently, the snap-element 244 and slot 260 are configured as a soft stop. After administration of the medicament into the subcutaneous layer of the skin of a patient, the user or practitioner can either dispose of the vascular access device 200 entirely or re-attach the sterile cap 290 while the vascular access device is still in the injection position and dispose of the vascular access device 200 and sterile cap 290. In some embodiments, the snap element 244 comprises the flat surface 246 and the sloped surface 249 and are configured to prevent the cammed slide 250 from rotationally traveling in the opposite direction, and thus preventing the vascular access device 200 from returning to the initial position

In some embodiments, release of the cammed slide 250 from the depression force will cause the cammed slide 250 to reversibly revert back to the initial position. The biasing force is directed in a distal direction relative to the cylindrical base 210 and the depression force is opposite the biasing force in a proximal direction relative to the cylindrical base 210. The biasing force and depression force cause longitudinal movement of the cammed slide 250, which also rotates between the initial position and aspiration position. Only upon application of a rotational force (by the hand of a practitioner or user) will the cammed slide 250 enter the injection position. The rotational force can be clockwise or counterclockwise. As previously stated, the cammed slide 250 is held in the injection position by the snap-element 244 of the cylindrical base 210 interdigitating with the slot 260 of the cammed slide 250. The cammed slide 250 cannot release from the injection position by longitudinal forces due to the cam path 232 being essentially a worm gear. To release from the injection position, the user must apply a rotational release force which, in some embodiments, is greater to or equal to the rotational force required to enter the injection position. Stated differently, the snap-element 244 and slot 260 are configured as a soft stop. Upon release from the injection position by applying the rotational release force, the biasing element causes the cammed slide 250 to enter the initial position, fully covering the needle cannula 80.

In operation, in the initial position as shown in FIG. 23, the needle cannula 80 is fully covered by the cammed slide 250 and the vascular access device 200 remains in the initial position due to the biasing element 280. Advancement of the vascular access device 200 against a surface, such as a rubber stopper of a vial, will apply the depression force necessary to fully expose the needle cannula 80 in the aspiration position. In the aspiration position, a practitioner or user can withdraw medicament from a vial.

In some embodiments, removal of the vascular access device 200 from the vial will cause the vascular access device 200 to advance to the injection position. In some embodiments, removal of the vascular access device 200 from the vial will cause the vascular access device 200 to return to the initial position. In some embodiments, application of rotational force will cause the vascular access device 200 to enter, and remain partially locked, the injection position.

In the injection position the needle cannula 80 is partially exposed by the distance D2 such that the needle cannula will only pierce the skin of a patient within the subcutaneous layer of the skin of a patient, but not penetrate into the muscle layer. In the injection position the practitioner or user can inject medicament into the subcutaneous layer of the skin of a patient. To dispose of the vascular access device 200, the sterile cap 90 can be placed back on and disposed of.

Referring back to FIGS. 21 and 22, in some embodiments, the cammed slide 250 further comprises markings 262 indicating to the user the direction of rotational motion the user must use to activate the vascular access device 200. In some embodiments, the cammed slide 250 further includes gripping indentations to assist in manipulation of the vascular access device 200.

As shown in FIGS. 26A through 26D, method of use of the vascular access device 200 comprises the steps of: advancing the cammed slide 250 of the vascular access device 200 against a stopper 50 of a vial 40 by applying the compression force against the stopper 50 such that the vascular access device 200 is advanced from the initial position to the aspiration position, withdrawing medicament from the vial 40, withdrawing the vascular access device 200 from the vial 40 which advances the vascular access device 200 to from the aspiration position to the injection position due to the biasing force, inserting the vascular access device 200 into the skin of a patient, injecting medicament into the subcutaneous layer of the skin of a patient, withdrawing the vascular access device from the skin of a patient, rotating the vascular access device 200 back to the initial position.

While the present disclosure has been shown and described with reference to certain exemplary embodiments thereof, it will be understood by those skilled in the art that various changes in form and details may be made therein without departing from the spirit and scope of the embodiments of the present disclosure. Also, the inner and/or the outer housing of the disinfection cap can be single shot molded, or made by other suitable process. Furthermore, any of the features or elements of any exemplary implementations of the embodiments of the present disclosure as described above and illustrated in the drawing figures can be implemented individually or in any combination(s) as would be readily appreciated by skilled artisans without departing from the spirit and scope of the embodiments of the present disclosure.

In addition, the included drawing figures further describe non-limiting examples of implementations of certain exemplary embodiments of the present disclosure and aid in the description of technology associated therewith. Any specific or relative dimensions or measurements provided in the drawings other as noted above are exemplary and not intended to limit the scope or content of the inventive design or methodology as understood by artisans skilled in the relevant field of invention.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has provided a description with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A vascular access device comprising:
a cylindrical base having a proximal end and a distal end, the proximal end having a proximal wall and an elongate tip extending from the proximal wall, the elongate tip having one or more longitudinal slots, the cylindrical base further having an outer cylindrical wall and an outer cavity between the outer cylindrical wall and the elongate tip, the outer cylindrical wall having a cutout extending from the distal end to the proximal wall;
a tab connected to the distal end of the outer cylindrical wall having a living hinge connecting the tab to the outer cylindrical wall, the tab configured to pivot about the living hinge, the tab covering the cutout of the cylindrical base in a closed position, the tab further comprising a crescent-shaped cam having a stem and a distal peak, the distal peak extending in a distal direction in the closed position; and,
a moveable collar disposed within the outer cavity, the moveable collar having a proximal end and a distal end, the distal end comprising one or more snap locks configure to travel within the one or more longitudinal slots, the moveable collar further comprising a clearance cutout extending from the distal end to a distance along the moveable collar, the clearance cutout having a cam-contact surface.

2. The device of claim 1, wherein the elongate tip comprises a proximal portion and a distal portion, the distal portion having an outer surface, the elongate tip further comprising an aperture extending therethrough, the one or more longitudinal slots extending on the proximal portion.

3. The device of claim 2, wherein a contact surface is formed between the proximal portion and distal portion of the elongate tip.

4. The device of claim 3, wherein a sterile cap is inserted over the elongate tip and is in contact with the contact surface.

5. The device of claim 1, wherein the living hinge further comprises one or more longitudinal protrusions configured to create a snap fit with corresponding slots disposed on the proximal wall.

6. The device of claim 1, wherein the proximal wall comprises a cavity for receiving a distal end of a vascular access device.

7. The device of claim 1, wherein the moveable collar further comprises a distal flange, the distal flange configured to abut the distal end of the outer cylindrical wall upon full advancement of the moveable collar within the cylindrical base.

8. The device of claim 1, wherein the moveable collar has an initial position, an aspiration position and an injection position.

9. The device of claim 8, wherein a needle cannula of the vascular access device is fully covered by the moveable collar in the initial position.

10. The device of claim 8, wherein a needle cannula of the vascular access device is fully exposed by a distance Df in the aspiration position.

11. The device of claim 10, wherein the distance Df is 6 mm.

12. The device of claim 8, wherein a needle cannula of the vascular access device is partially exposed by a distance Dn in the injection position.

13. The device of claim 12, wherein in the distance Dn is 4 mm.

14. The device of claim 8, wherein advancement of the moveable collar from the initial position causes the cam-contact surface of the moveable collar to abut against the crescent-shaped cam of the tab, causing the tab to pivot outwardly about the living hinge.

15. The device of claim 14, wherein depressing of the tab causes the moveable collar to advance to the injection position.

16. A vascular access device comprising:
a cylindrical base having a proximal end, a distal end, a proximal wall and an elongate tip extending from the proximal wall, elongate tip further comprising an aperture extending therethrough, the cylindrical base having an outer cylindrical wall and a cavity defined by the elongate tip, outer cylindrical wall and the proximal wall, the cylindrical base further having a cam path disposed on an outer surface of the outer cylindrical wall, the distal end having a snap-element;
a cammed slide disposed over the cylindrical base having a proximal end, a distal end and an inner surface, the distal end having a distal wall, the inner surface having a pegextending inwardly and a slot, the peg configured to travel within the cam path; and,
a biasing element disposed between the distal wall of the cammed slide and the proximal wall of the cylindrical base, the biasing element configured to apply a biasing force;
wherein the peg advances along the cam path between at least three points, a first point defining an initial position, a second point defining an aspiration position and a third point defining an injection position, wherein a needle cannula is fully covered in the initial position, the needle cannula is fully exposed by a distance D1 in the aspiration position and the needle cannula is partially exposed by a distance D2 in the injection position.

17. The device of claim 16, wherein the proximal wall comprises a cavity for receiving a distal end of a vascular access device.

18. The device of claim 16, wherein the distal wall of the cammed slide comprises an inner collar extending in a proximal direction.

19. The device of claim 16, wherein the peg is positioned at a right angle plane relative to the slot and the peg is located proximally to the slot.

20. The device of claim 16, wherein the peg can advance in a clockwise or counterclockwise direction.

21. The device of claim 16, wherein the snap-element comprises a flat surface and a sloped surface opposite the sloped surface.

22. The device of claim 16, wherein the distance D1 is 6 mm and the distance D2 is 4 mm.

23. The device of claim 16, wherein the biasing element is configured to continuously hold the cammed slide in the initial position such that the needle cannula is covered.

24. The device of claim 16, wherein depression of the cammed slide in a proximal direction relative to the cylindrical base with a depression force greater than the biasing force causes the cammed slide to reversibly advance to the aspiration position.

25. The device of claim 16, wherein depression of the cammed slide in a proximal direction relative to the cylindrical base with a depression force greater than the biasing force causes the cammed slide to non-reversibly advance to the aspiration position.

26. The device of claim 25, wherein withdrawing the depression force causes the cammed slide to enter the injection position.

27. The device of claim 26, wherein the cammed slide is held in the injection position by the snap-element of the cylindrical base interdigitating with the slot of the cammed slide.

28. The device of claim 27, wherein the cammed slide returns to the initial position upon application of a rotational release force.
